(19) United States
(12) Patent Application Publication
(10) Pub. No.: US 2011/0027842 A1
(43) Pub. Date: Feb. 3, 2011

(54) Method for Producing Monosaturated Glycerides

(75) Inventors: Per Munk Nielsen, Hilleroed (DK); Steffen Ernst, Broenshoej (DK); Kim Borch, Birkeroed (DK); Hans Christian Holm, Hellerup (DK); Yee Hon Seng, Malaysia (MY); David William Cowan, Buckinghamshire (GB)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/936,251

(22) PCT Filed: Mar. 25, 2009
(86) PCT No.: PCT/EP2009/053513

§ 371 Date: Oct. 4, 2010

Related U.S. Application Data
(60) Provisional application No. 61/043,187, filed on Apr. 8, 2008.

(30) Foreign Application Priority Data
Apr. 7, 2008 (EP) 08154158.3

(51) Int. Cl. C12P 7/64; C07C 57/02

(52) U.S. Cl. 435/134; 554/1

(57) ABSTRACT

A process for producing a glyceride product which is enriched in monounsaturated fatty acids relative to the starting glyceride comprising the steps: (a) alcoholysis of triglycerides employing lipolytic enzymes selective for saturated fatty acids and/or lipolytic enzymes selective for the 1-position, the 3-position or both positions in a glyceride; and (b) separation of fraction A which is enriched in saturated fatty acid esters from fraction B which is enriched in monounsaturated glycerides.

### FIELD OF THE INVENTION

The invention relates to the field of glycerides. It relates to the manufacturing of glycerides by employing lipolytic enzymes. More particularly, the invention relates to a process for producing monounsaturated glycerides by using specific lipolytic enzymes.

### BACKGROUND OF THE INVENTION

Diets with high levels of saturated fats are known to raise blood cholesterol and to increase the risk of cardiac vascular diseases. It is therefore desirable to decrease the amount of saturated fats and to increase the amount of unsaturated fats in consumer products.

Some reports have been published disclosing processes for producing polyunsaturated fats. WO 95/24459 (Norsk Hydro A/S) describes a process applied to fish oils where a fraction enriched in polyunsaturated glycerides is separated from a fraction with saturated fatty acids and monounsaturated glycerides. They do not recombine fractions to form triglycerides but proceed with alcoholysis until essentially all fatty acids are esterified. U.S. Pat. No. 6,905,850 B2 (Nippon Suisan Kaisha, Ltd) also relates to fish oils. It describes a process for producing triglycerides having polyunsaturated fatty acids in the 2-position and medium-chain saturated fatty acid residues having the carbon number of 8, 10 or 12 at the 1- and 3-positions.

A reduction of polyunsaturated fatty acids is know to increase the stability of some consumer products such as e.g. frying medium and it is therefore desirable to obtain products high in monounsaturated fatty acids and low in saturated and/or polyunsaturated fatty acids.

The Malaysian Palm Oil Board has described a process based on partial fractionation in which they start with palm olein and achieve an end product with 60% monounsaturates (M. R. Ramli, W. L. Siew, K. Y. Cheah (2008) Properties of High-Oleic Palm Oils Derived by Fractional Crystallization Journal of Food Science 73 (3), C140-C145 doi:10.1111/j.1750-3841.2007.00657.x). They state, however that the goal of achieving an end product with 80% monounsaturates is still years of research away, and thus, there is still a strong desire to develop processes to produce fractions enriched in monounsaturated glycerides.

### SUMMARY OF THE INVENTION

In a first aspect, the invention relates to a process for producing a glyceride product which is enriched in monounsaturated fatty acids relative to the starting glyceride comprising the steps: (a) alcoholysis of triglycerides employing lipolytic enzymes selective for saturated fatty acids and/or lipolytic enzymes selective for the 1-position, the 3-position or both positions in a glyceride; and (b) separation of a fraction A which is enriched in saturated fatty acid esters from a fraction B which is enriched in monounsaturated glycerides.

In a second aspect, the invention relates to use of the fraction A1 and optionally the fraction A3 both enriched in saturated fatty acid esters for producing biodiesel, surfactant, or high purity grade chemicals.

In a third aspect, the invention relates to use of glycerides enriched in monounsaturated fatty acids for producing consumer products and/or fried food products preferably edible oil, consumer oil, margarine, shortenings, frying oil, battered fried products, baked products like bread, cake, cookies, biscuits or snack foods such as e.g. chips and French fries.

In a fourth aspect, the invention relates to a glyceride product obtainable by the process comprising at least 70 mole %, at least 75 mole %, at least 80 mole %, at least 85 mole %, at least 90 mole %, at least 95 mole %, at least 96 mole %, at least 97 mole %, at least 98 mole %, at least 99 mole %, or 100 mole % monounsaturated fatty acids.

In a fifth aspect, the invention relates to a glyceride product obtainable by the process comprising at least 70 mole %, at least 75 mole %, at least 80 mole %, at least 85 mole %, at least 90 mole %, at least 95 mole %, at least 96 mole %, at least 97 mole %, at least 98 mole %, at least 99 mole %, or 100 mole % triglycerides.

### BRIEF DESCRIPTION OF THE FIGURES

FIG. 1 shows Process for producing a glyceride product by condensation

FIG. 2 shows Distillative enrichment of alcohol esters

### DEFINITION OF TERMS

The terms to be defined below are shown in capitals and have been listed alphabetically:

ALCOHOLYSIS is the reaction between an alcohol and a glyceride such as an oil or fat. If the alcohol concerned is ethanol the alcoholysis can also be referred to as ethanolysis, if methanol is employed the alcoholysis can also be referred to as ‘methanolysis’, etc.

BIODIESEL is defined as esters of long chain fatty acids derived from renewable feed stocks and C₁-C₃ monohydric alcohols. Examples of such renewable feed stocks are vegetable oils and animal fats. In the context of the present invention long chain fatty acids may be defined as fatty acid chains with a length of between 10 and 22 carbon atoms.

CONVERSION is defined as the molar fraction of fatty acids in the glycerides structure of the raw material that have been reacted by the enzyme catalyzed reaction. This can be measured by mol. For transesterification of glycerides with ethanol: Conversion=FAEE/FAIG, where FAEE=mol Fatty Acid Ethyl Ester after reaction and FAIG=mol Fatty Acids in glycerides before reaction. For hydrolysis of glycerides: Conversion=(FFA_{end}−FFAₛₜₐᵣₜ)/FAIG, where FFA_{end}=mol Free Fatty Acids after reaction, FFAₛₜₐᵣₜ=mol Free Fatty Acids in raw material before the reaction, and FAIG=mol Fatty Acids in glycerides before reaction.

CRYSTALLISATION is used here to describe solid/liquid separation processes based on differences in melting points, i.e. carried out at a temperature where some compounds of a mixture are solid and some are not. Crystallization is also referred to as thermal fractionation and both terms are used interchangeably.

DEODORISATION is essentially a steam distillation under vacuum.

DISTILLATION is the process of heating a liquid to its boiling point and condensing and collecting the vapor in liquid form.

ESTERIFICATION is the reaction between a fatty acid and an alcohol leading to an ester and water.

EVAPORATION is a process step converting at least one component to the vapor form. Evaporation comprises specific forms such as distillation and deodorization.

HYDROLYSIS is the reaction between an ester and water and is the reversible reaction of esterification.

FATTY ACID DISTILLATE is the condensate resulting from a vapour scrubbing process during the vacuum stripping of triglyceride oils which latter process is used for the physical removal of free fatty acids and for the deodorisation of triglyceride oils. In addition to FFA or FFA esters, the fatty acid distillate contains unsaponifiables such as but not limited to tocopherols and sterols.

FATTY FEED is a general name for raw materials containing fatty acid moieties. These can be glycerides such as monoacylglyceride, also referred to as monoglyceride, diglycerides, triglycerides and phosphatides but free fatty acids and even soaps can form part of the fatty feed.

FFA is the standard abbreviation of Free Fatty Acids.

OLEIN of oil or a fat product is the low-melting fraction obtained by solid/liquid separation of the product at a temperature where part of the content is solidified.

MEMBRANE SEPARATION designates processes, by which liquid/liquid separation of different molecular species is secured by semi-permeable membranes.

MOLECULAR DISTILLATION is distillation in high vacuum, intended to make possible use of low temperatures to protect heat-labile compounds.

STRIPPING, also referred to as vacuum stripping when carried out at subatmospheric pressure, is a process that causes the most volatile constituents of a mixture to vaporize when a gas is blown through the mixture.

THERMAL FRACTIONATION is another term for crystallization.

TRANSESTERIFICATION is the reaction between a glyceride having R1 and a fatty acid having R2 whereby the R-groups is exchanged leading to a glyceride having R2 and a fatty acid having R1.

### DETAILED DESCRIPTION OF THE INVENTION

An object of the present invention is to provide a high efficient process for producing high purity glyceride products which is enriched in monounsaturated fatty acids relative to the starting glyceride. The aim is furthermore to generate other high purity products such as e.g. fatty acid esters which may be saturated for biodiesel production or unsaturated, in particular monounsaturated which may be reused in the process of the invention. It is asserted that glyceride, fatty acid, fatty acid ester, glycerol, and alcohol products obtained by said process have a high purity chemical grade or high purity food grade.

Lipolytic enzymes have successfully been used as biocatalysts in the fractionation of fatty acids and other lipids. The ability of certain lipolytic enzymes to discriminate against or prefer particular substrates have been utilized for the selective enrichment of fatty acids or their esters from natural fats and oils in many types of reactions such as e.g. hydrolysis, esterification, interesterification and transesterification. Whereas the reaction rate is dependent on the type of reaction and factors such as temperature, pressure and excess and/or depletion of reactants, the specificity of the enzyme generally remains the same. An example may be G. candidum lipase which has a distinct preference for C18 acyl moieties having cis-9 or cis-9, cis-12-bonds and discriminates against cis-13-22:1 both in hydrolysis of triacylglycerol and in esterification of fatty acids with n-butanol.

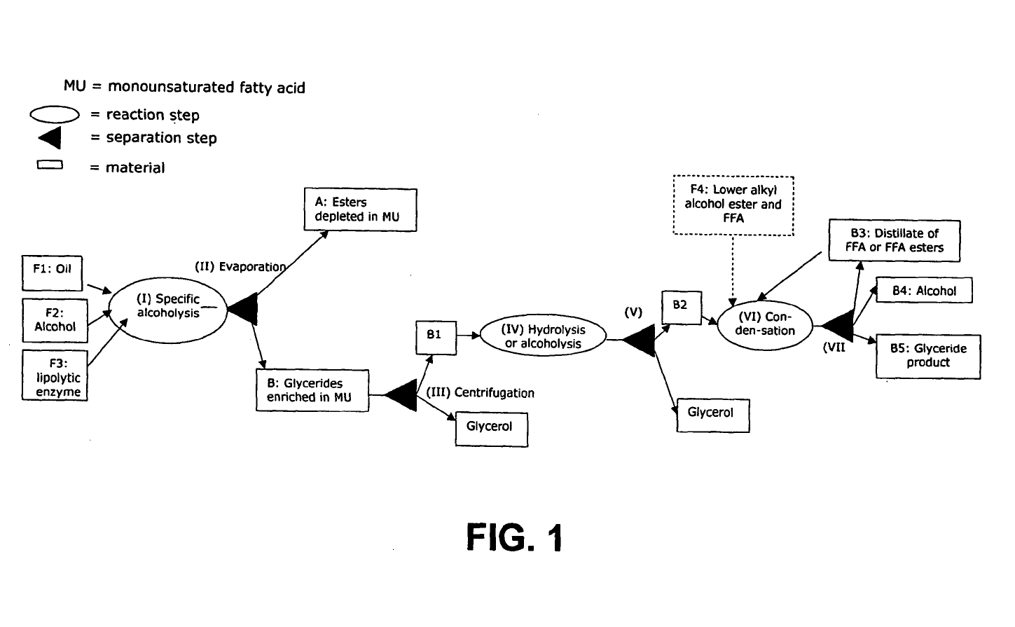

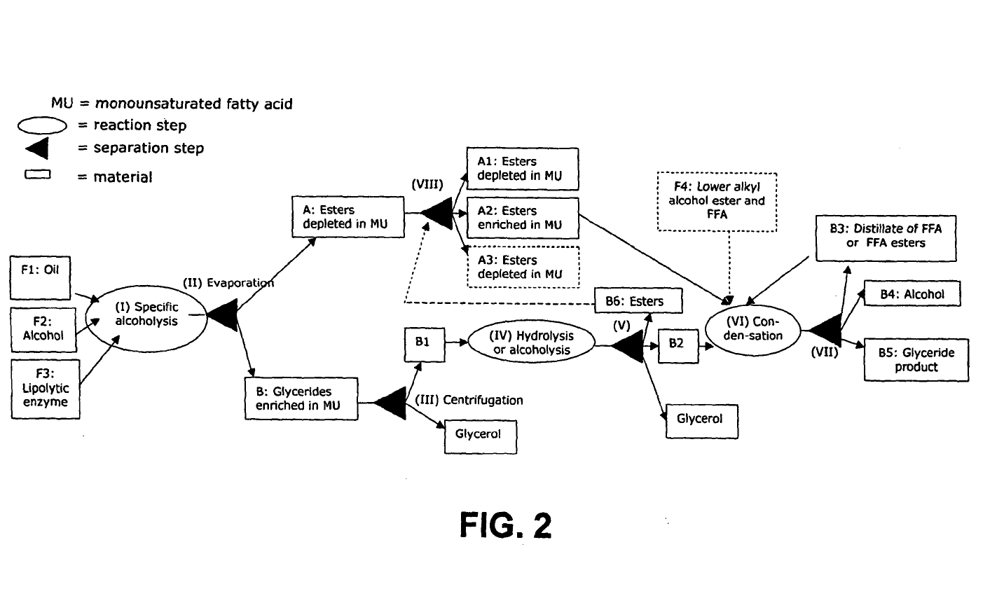

The FIGS. 1 and 2 have been included for illustration purposes alone and should in no way be construed as limiting the invention. References have been made in the text to the figures by applying the nomenclature used in the figures.

FIG. 1 shows the specific alcoholysis (I) of oil (F1)+alcohol (F2)+specific lipolytic enzyme (F3). Two fractions are separated by evaporation (II) in which one is esters depleted in monounsaturated esters (A) and the other is glycerides enriched in monounsaturated fatty acids (B). Fraction B may, depending on the oil (F1) and the starting triglyceride contain various amounts of glycerol, mono-, di- and triglycerides and are separated by centrifugation (III) to obtain a glyceride fraction depleted of glycerol (B1). This fraction and/or fraction B may optionally be submitted to one or more rounds of alcoholysis or hydrolysis (IV) followed by separation (V) by evaporation and/or centrifugation to separate out more glycerol resulting in a fraction further enriched in monounsaturated glycerides (B2). Fraction B2 is then re-esterificated by condensation (VI) by adding monounsaturated fatty acid or fatty acid esters. The following separation (VII) results in a glyceride product (B5) enriched in monounsaturated fatty acids relative to the starting glyceride. Other fractions such as an alcohol (B4) and a distillate of fatty acid or fatty acid esters (B3) may also be isolated. It is envisaged that the distillate (B3) may be recycled to the condensation step (VI).

In some embodiments the invention relates to a process for producing a glyceride product which is enriched in monounsaturated fatty acids relative to the starting glyceride comprising the steps: (a) alcoholysis of triglycerides employing lipolytic enzymes selective for saturated fatty acids and/or lipolytic enzymes selective for the 1-position, the 3-position or both positions in a glyceride; and (b) separation of a fraction A which is enriched in saturated fatty acid esters from a fraction B which is enriched in monounsaturated glycerides.

In a preferred embodiment the enzyme used in the alcoholysis step is specific for saturated fatty acids. The specificity of some lipolytic enzymes specific for saturated fatty acids is described in: Heldt-Hansen et al: “A new immobilized positional nonspecific lipase for fat modification and ester synthesis”, ACS Symposium Series, Biocatalysis In Agricultural Biotechnology, vol. 389, 1989, pp. 158-172 showing that Candida antarctica A lipase is 4.3 times more active in acidolysis of tricaprylin using a saturated fatty acid (lauric acid) vs. an unsaturated fatty acid (oleic acid). This implies that it will also have a preference for saturated acids in triglycerides; and in Joshi and Dhah, Acta Microbiologica Hungarica, vol. 34, pp. 111-114, 1987 showing that Fusarium oxysporum lipase selectively hydrolysed the saturated fatty acids of three different substrates: cotton seed oil, ground-nut oil and fungal oil from Fusarium.

Lipolytic enzymes selective for saturated fatty acids independently of position can be identified by a test using two homogeneous triglyceride—triple-saturated and triple-monounsaturated and compare the reaction rate with ethanol of the enzyme on these two substrates at identical conditions—either in separate containers or in a mixture. They can also be found by a test using two ethyl esters (saturated/monounsaturated) and react with glycerol or homogenous triglyceride (three identical fatty acids) under vacuum to remove ethanol, and compare the reaction rate with each of the two ethyl esters. Specifically, the saturated fatty acid is palmitic, the monounsaturated fatty acid is oleic, and the criterion for whether an enzyme is ‘saturation-specific’ is to be 2 times more active on the saturated substrate at a conversion which falls in the range of 0.05 to 0.50. Enzymes identified as ‘saturation specific’ by either of these two tests are useful in this invention.

Lipolytic enzymes selective for saturated fatty acids in a given oil substrate can be found by performing enzymatic ethanolysis of the oil and analyzing the reaction products by GC after appropriate sample preparation as described by Moreira et al (Energy and Fuels, vol. 21, pp 3689-3694, 2007). This will allow determination of each of the ethyl-esters. By comparing this composition with the fatty acid distribution of the starting material, one can identify if the reaction rate constant of saturated fatty acids was 1.5 times, preferably 2 times or 3 times that on unsaturated fatty acids at a conversion which falls in the range of 0.05 to 0.50. The reaction rate constant is found by dividing the reaction rate (i.e. the amount of formed fatty acid esters per time unit) by the starting concentration of that particular fatty acid in the substrate.

In a preferred embodiment the enzyme used in the alcoholysis step is 1,3 specific. The use of an enzyme in the alcoholysis of palm oil which is 1,3 specific will result in predominantly saturated fatty acid esters because Palm oil almost exclusively (˜85%) carries unsaturated fatty acids in the 2-position. The specificity of some 1,3 specific lipolytic enzymes is described in: Shen et al, JAOCS vol 83, pp 923-927 (2006) which uses Novozyme 435 (an immobilised form of Candida Antarctica Lipase B) for regioselective ethanolysis of triacylglycerols with high unsaturated fatty acid content; Rogalski et al Chirality, vol. 5, pp. 24-30 (1993) demonstrate a number of lipases with quite strict 1,3 specificity at the experimental conditions—among them Candida Antarctica B lipase; Rhizomucor miehi lipase and lipolase from Humicola; and Ghazali et al el. JAOCS vol 72, pp. 633-639 (1995) who show transesterification of palm olein with 1,3 specific lipases—including several of those mentioned above. Other examples of enzymes that have been classified as 1,3 specific are: Pseudomonas fluorescense (Lipase AK from Amano) and Burkholderia cepacia (Lipase PS from Amano), Candida rugosa (Lipase AYS from Amano), Rhizopus oryzae (Lipase F-AP 15, from Amano), Penicillium camemberti (Lipase G from Amano), Rhizopus javenicus (Lipase M from Amano), Penicillium roquefortii (Lipase R from Amano).

Lipolytic enzymes selective for the 1-position, the 3-position or both positions can be identified by a method described by Rogalski et al (Chirality, vol. 5, pp 24-30, 1993). Briefly, triolein is subjected to enzymatic hydrolysis in a titration apparatus and the reaction is stopped at 6% conversion. The reaction products are analyzed by HPLC; which can quantify the formed 1,3- and 1,2 diglycerides. The relative amount of these formed indicate the positional specificity of the enzyme. When less than 5%, preferably less than 3% or 1% of the deacylation occurs in the sn-2 position, the enzyme can be termed selective for the 1,3 position.

In some embodiments the invention relates to a process further comprising a step (c) alcoholysis or hydrolysis of fraction B or a sub fraction thereof employing either (i) lipolytic enzymes selective for saturated fatty acids and/or lipolytic enzymes selective for the 1-position, the 3-position or both positions in a glyceride, or (ii) a lipolytic enzyme which is selective for monoglyceride. In step (IV), the fraction B is further processed by a second enzymatic step to degrade the glycerides: In alcholysis to generate alcohol-esters, glycerol and residual glycerides; or in hydrolysis to generate free fatty acids, glycerol and residual glycerides.

Monoglyceride specifik lipolytic enzymes may be selected from lipases isolated from mammalian tissue such as e.g. monoacylglycerol hydrolyzing enzyme of rat adipocytes, rat liver microsomal monoacylglycerol lipase, monoacylglycerol lipase in human erythrocytes, or from a bacterial strain such as e.g. monoacylglycerol lipase from Pseudomonas sp. LP7315, or monoacylglycerol lipase from the moderately thermophilic Bacillus sp. H-257.

The objective of step (IV) is to form free glycerol, and optionally allow it to be separated in order for the stoichiometry of step (VI) to favor triglyceride formation. It is preferred to use a monoglyceride-specific lipase in step (IV), as this would give the most effective release of glycerol from fraction B or B1 which are mixtures of primarily mono- and diglycerides. One such is exemplified by Sakiyama et al (J. Bioscience and Bioeng. vol 91, pp 27-32, 2001), who isolated and characterized a lipase from Pseudomonas sp. LP7315 and demonstrated that it has a high selectivity of monoacylglycerides over diacylglycerides. The activity on monoglycerides of olein, stearin, palmin and linolein was approximately equal, and the enzyme is stable at 65° C. suitable for the processes envisioned here. Also, Imamura and Kitarura isolated a monoglyceride-specific lipase from Bacillus sp. H257 (J. Biochem., vol. 127, pp 419-425, 2000). Either of these enzymes would be useful in step (IV) of certain embodiments of the invention.

The product mixture resulting from either alcoholysis or hydrolysis (IV) is separated in step (V). Separation can be a liquid/liquid separation such as e.g. centrifugation to separate glycerol from the other products which may proceed to step (VI). Alternatively, the separation step (V) is arranged as two sequential unit operations: a vapor/liquid separation such as deodorisation or molecular distillation, to isolate alcohol esters or free fatty acids (fraction B6, which optionally may be added to the step VIII for further separation into sub fractions of esters enriched in monounsaturated or saturated fatty acids, respectively), and a liquid/liquid separation to isolate glycerol such as e.g. centrifugation, decantation and membrane seperation.

In some embodiments the invention relates to a process further comprising a step (d) removal of glycerol from the glyceride fractions by methods of centrifugation, decantation, or membrane separation. Such glycerol may in certain embodiments be recycled to the step of re-esterification by condensation (VI) with monounsaturated fatty acid esters to the extent needed to get stoichiometric conversion to triglycerides. In other embodiments, such glycerol may be discarded or sold for other uses. The glycerol removal may be performed on fraction B directly after step III or after a further separation step (V).

In certain embodiments the separation steps (III) and/or (V), may include a water wash prior to centrifugation, thereby augmenting the separation of glycerol from the glycerides. Furthermore, the method of membrane separation may be used to separate glycerol from the glycerides. This is described by Dubé et al (Bioresource technology, vol:98 iss:3 pp:639-647, 2007), who uses a membrane reactor in production of Fatty acid methyl esters and separates unreacted glycerides from the glycerol product using a carbon membrane.

In some embodiments the invention relates to a process, wherein the triglycerides comprise at least 30%, at least 35%, at least 40%, at least 45% or at least 50% monounsaturated fatty acids.

In some embodiments the invention relates to a process, wherein the triglycerides are having at least 50%, at least 55%, at least 60%, at least 65%, at least 70%, at least 75%, or at least 80% monounsaturated fatty acid residues in the 2-position.

In some embodiments the invention relates to a process, wherein the source of triglycerides is palm oil; peanut oil; soybean oil; rapeseed oil; sunflower oil; olive oil; beef tallow; butterfat; cocoa butter; pork lard; poultry fat or their corresponding olein. It is preferred to use palm oil or preferably palm olein as starting material (palm olein is an olein-enriched fraction of palm oil obtained by thermal fractionation). Methods of obtaining oleins are well-known and described e.g. ‘Introduction to Fats and Oil Technology’, eds. O'Brien, Farrr and Wan, AOCS Press, 2000 chapter 11.

In some embodiments the invention relates to a process, wherein the alcoholysis is performed by conversion of the triglyceride with a lower alkyl alcohol, preferably a C1-C3 alcohol, and more preferably ethanol. By using ethanol as the alcohol for alcoholysis would improve the food utility of the products.

It is anticipated that the lipolytic enzyme specificities mentioned above (both saturated/unsaturated specificity as well as 1,3 specificity) will be high at a low degree of conversion which will decrease concurrently with the depletion of the preferred substrate and the simultaneously increase of the less preferred substrate. Hence, it is preferred to run the reaction at low conversion in order to secure the highest possible specificity. It is an advantage in certain embodiments of the invention to make the best utility of all reaction products, even at low conversion rates of alcoholysis.

In some embodiments the invention relates to a process, wherein the conversion in alcoholysis to fatty acid esters is below 5%, below 10%, below 15%, below 20%, below 25%, below 30%, below 35%, below 40%, below 45% or below 50%.

In some embodiments the invention relates to a process, wherein the conversion in alcoholysis to fatty acid esters is at least 40%, at least 45%, at least 50%, at least 55%, at least 60%, at least 65%, or at least 70%.

In some embodiments the invention relates to a process, wherein the lipolytic enzyme selective for saturated fatty acids is selected from Candida antarctica lipase A, Fusarium oxysporum lipase, and variants thereof.

In some embodiments the invention relates to a process, wherein the lipolytic enzyme selective for the 1-position, the 3-position or both positions is selected from Candida antarctica B lipase, Chromobacterium viscosum, dog gastric lipase, dog pancreatic lipase, Fusarium solani cutinase lipase, guinea pig pancreatic lipase, human gastric lipase, Humicola lanuginosus lipase, human pancreatic lipase, lipoprotein lipase, Mucor miehei lipase, Pseudomonas aeruginosa lipase, Penicillium camemberti lipase, Pseudomonas fluorescens lipase, Pseudomonas glumae lipase, porcine pancreatic lipase, Penicillium simplicissimum lipase, Rhizopus arrhizus lipase, rabbit gastric lipase, Fusarium heterosporum lipase, Candida rugosa lipase, and variants thereof.

In some embodiments the invention relates to a process, wherein the glyceride product comprises long-chain fatty acids, preferably having a carbon number of at least 14, at least 16, at least 18 or any combination thereof.

In some embodiments the invention relates to a process, wherein the separation method is selected from deodorization, distillation, evaporation, or any combination thereof. Any unreacted fatty acid esters or free fatty acids, as well as released alcohol may be removed as the volatile fraction by deodorization, evaporation or distillation. This volatile fraction can further be separated into alcohol (optionally for reuse in step (I)) and the unreacted free fatty acid or fatty acid ester, which may be reused in step (VI). Deodorisation is essentially a steam distillation under vacuum and is well known in the art. A deodorizer may be operated at 0.15 mbar, 225° C. with steam dosage of 0.20% to 0.25% w/w per hour. Other modes of operation are known in the art, see e.g. ‘Introduction to Fats and Oil Technology’, Eds O'Brien, Farrr and Wan, AOCS Press, 2000 chapter 13.

The methods of distillation and evaporation are also known in the art. Evaporation units for oils are usually vapor distillation units, called deodorizers. For step (VIII) it is an embodiment to use distillation under high vacuum to minimize thermal damage. It is in certain embodiments of the invention preferred to use a system with multiple equilibrium stages to achieve a good separation. Other preferred embodiments include Falling film Molecular Distillators operated at pressures of 0.001 to 10 mmHg and temperatures of 140° C. to 200° C., or Centrifugal Molecular Distillators which can operate at pressures around 0.001-10 mmHg and temperatures of 160° C. to 240° C. (both of these modes are described in detail in Batistella et al, Appl. Biotechn., vol. 98, 1149-1159, 2002). It is possible to use direct or indirect heating, and it is possible to operate in batch and/or continuous operation.

It is also an embodiment, to operate separation step II in such a manner that only ethyl-palmitate (and more volatile components) are separated with the distillate and ethyl-stearate, ethyl-oleate and ethyl-linoleate remains in the concentrate along with the glycerides. In certain embodiments of the invention the pressure and the temperature are selected achieve the best possible separation between ethyl-palmitate and other ethyl esters.

FIG. 2 comprises and shows in addition to FIG. 1 a further separation of fraction A (VIII) into esters depleted in monounsaturated fatty acids (A1), esters enriched in monounsaturated fatty acids (A2), and optionally esters depleted in monounsaturated fatty acids which is different from sub fraction A1 (A3). Depending on the content of fatty acids in the starting glyceride, further sub fractions may be generated. This separation may be carried out by distillation or alternatively by membrane separation or crystallization or supercritical extraction.

In the case of e.g. palm oil or palm olein a separation by distillation may be set up to obtain almost pure palmitic acid ester. The fatty acid esters formed in alcoholysis will primarily consist of palmitic, stearic, oleic and linoleic acids. As the boiling points are such that stearic, oleic, and linoleic acids separate together (see Batistella et al, ‘Mathematical development for scaling up of molecular distillators: strategy and test with recovering carotenoids from palm oil, 16^{th} Eur. Symp. on Comp. Aided Proc. Eng. and 9^{th} Int. Symp. on Process Systems Eng., Eds. Marquardt and Pentelides, Elsevier, 2006), one may arrange the distillation so one fraction is primarily palmitic acid ester while the other is a mixture of stearic, oleic and linoleic acid esters. Stearic acid is present only in small amounts (<5%) in palm olein and the stearic/oleic/linoleic acid ester fraction will constitute a fraction substantially enriched in monounsaturated fatty acid, while the palmitic acid ester fraction may be made so pure in palmitic acid ester that it may gain a premium value such as e.g. for synthesis of surfactants or other chemicals. If desired the separation may be set up to obtain fractions of each fatty acid ester.

It is envisaged that the esters enriched in monounsaturated fatty acids (A2) may be recycled to the condensation step (VI), alternatively, this fraction (A2) may be combined with glycerol to form glyceride products enriched in monounsaturated fatty acids (not shown in the figures). In addition, during the reaction step of alcoholysis (IV) esters which may be depleted in monounsaturated fatty acids are formed (B6) these may also be separated and recycled and together with the first fraction of esters depleted in monounsaturated fatty acids (A) they may enter the separation step (VIII). Alternatively, the fatty acids or fatty acid esters that may be used for the condensation step (VI) may be provided from an external source (F4). It may be a hydrolysate or alcoholysate of a vegetable oil such as e.g. sunflower oil, peanut oil, rapeseed oil, soybean oil, olive oil or alternatively from modified varieties thereof enriched in monounsaturates and/or reduced in polyunsaturates.

In some embodiments the invention relates to a process, wherein the fraction A enriched in saturated fatty acid esters is further purified to obtain a sub fraction A1 which relative to fraction A is enriched in saturated fatty acid esters, a sub fraction A2 which relative to fraction A is enriched in monounsaturated fatty acid esters, and optionally a sub fraction A3 which relative to fraction A is enriched in saturated fatty acid esters and which is different from sub fraction A1. The separation of alcohol ester fraction from glycerides (II) may be done by evaporation or deodorization. In certain embodiments of the invention step (II) and (VIII) may be combined in a single unit operation, which separates the alcohol esters from glycerides and further separates the alcohol esters into several sub fractions.

In some embodiments the invention relates to a process, wherein the sub fraction A2 is even further purified to obtain a sub fraction A2* which is even more enriched in monounsaturated fatty acid esters. In the separation step of esters (VIII), a process may be devised in which the esters depleted in monounsaturated fatty acids (A) and which may be enriched in saturated fatty acids is further separated in an even more enriched saturated fatty acid-ester and a fraction which contains the unsaturated FA-esters. This separation can be carried out by distillation or alternatively by membrane separation, crystallization, or supercritical extraction (e.g. Crampon, J. Supercritical fluids, vol. 16, 11-20, 1999).

In some embodiments the invention relates to a process, wherein the sub fraction A1 is essentially a single molecular species.

In some embodiments the invention relates to a process, wherein sub fraction A1 essentially being ethyl-palmitate; sub fraction A2 essentially being ethyl-oleate and sub fraction A3 essentially being ethyl-stearate.

In some embodiments the invention relates to a process, wherein the sub fraction A1 is at least 80%, at least 85%, at least 90%, at least 95%, at least 96%, at least 97%, at least 98%, at least 99% or 100% ethyl-palmitate.

In some embodiments the invention relates to a process, wherein the fraction (B) enriched in monounsaturated glycerides and/or any sub fractions derived thereof is re-esterified with a composition rich in monounsaturated fatty acid present as esters or free fatty acids, to produce a glyceride product having at least 70%, at least 75%, at least 80%, at least 85%, at least 90%, at least 95%, at least 96%, at least 97%, at least 98%, at least 99% or 100% monounsaturated fatty acids.

In some embodiments the invention relates to a process, wherein the re-esterification is enzymatic.

In some embodiments the invention relates to a process, wherein the monounsaturated fatty acid esters for re-esterification are obtained from the sub fraction A2, the sub fraction A2*, or a hydrolysate, a distillate or alcoholysate of a vegetable oil.

In some embodiments the invention relates to a process, wherein the vegetable oil is selected from sunflower oil, peanut oil rapeseed oil, soybean oil, olive oil or alternatively from modified varieties thereof enriched in monounsaturates and/or reduced in polyunsaturates.

In some embodiments the invention relates to a process, wherein the content of triglycerides in the glyceride product is at least 70%, at least 75%, at least 80%, at least 85%, at least 90%, at least 95%, at least 96%, at least 97%, at least 98%, at least 99% or 100%. In certain embodiments of the invention it is the goal to achieve high, but less than 100% conversion to triglycerides.

In some embodiments the invention relates to a process, wherein the re-esterification further comprises a step to remove volatiles such as released alcohols or unreacted esters and fatty acids.

In some embodiments the invention relates to a process, wherein the step to remove volatiles is selected from evaporation, distillation, and deodorization.

In some embodiments the invention relates to a process, wherein the unreacted esters or fatty acids are reused for re-esterification.

In some embodiments the invention relates to use of the fraction A1 and optionally the fraction A3 both enriched in saturated fatty acid esters for producing biodiesel, surfactant, or high purity grade chemicals. For example, rather pure ethanol ester biodiesel obtained by certain embodiments of the invention may be used to blend into biodiesel produced by waste sources, which is presumed to be of variable quality and in need of a stable blending agent to obtain a consistent quality.

In some embodiments the invention relates to use of glycerides enriched in monounsaturated fatty acids for producing consumer products and/or fried food products preferably edible oil, consumer oil, margarine, shortenings, frying oil, battered fried products, baked products like bread, cake, cookies, biscuits or snack foods such as e.g. chips and French fries. It is envisaged that the glycerides may be the fractions B1, B2, B5, or any combinations thereof. The glyceride product enriched in monounsaturated fatty acids according to certain embodiments of the invention is considered healthier oil for nutritional purposes. In particular, oils rich in monounsaturated fatty acids are considered healthy and useful as frying oils due to their high stability.

In some embodiments the invention relates to a glyceride product obtainable by the process comprising at least 70 mole %, at least 75 mole %, at least 80 mole %, at least 85 mole %, at least 90 mole %, at least 95 mole %, at least 96 mole %, at least 97 mole %, at least 98 mole %, at least 99 mole %, or 100 mole % monounsaturated fatty acids of the total fatty acids in the glycerides.

In some embodiments the invention relates to a glyceride product, further comprising less than 5%, less than 4%, less than 3%, less than 2%, less than 1% of saturated fatty acids of the total fatty acids in the glycerides.

In some embodiments the invention relates to a glyceride product obtainable by the process comprising at least 70 mole %, at least 75 mole %, at least 80 mole %, at least 85 mole %, at least 90 mole %, at least 95 mole %, at least 96 mole %, at least 97 mole %, at least 98 mole %, at least 99 mole %, or 100 mole % triglycerides.

### EXAMPLES

The present invention is further described by the following examples that should not be construed as limiting the scope of the invention.

### Example 1

### Specificity Study for Candida Antarctica Lipase A

The enzyme Candida Antarctica lipase A-Novozym 735-N735 (batch: LDN00026) with an activity of 6KLU was used. The substrates: Palm Stearine (PS); Soybean Oil (SBO); and High Oleic Oil, all purchased from Sigma, was tested

### A. Fat Hydrolysis

1. Weight 200 g of oil into a 500 ml empty screw cap flask.
2. Add 35 g of water into the same flask and keep at oven temperature of 70° C. for one hour.
3. Treat by high shear mixing (IKA Ultra Turrax T25) at 24,000 rpm for 90 seconds.
4. Transfer 100 g of the emulsion to a 250 ml empty screw cap flask.
5. Add in 600 ppm (51 μl) of Novozym 735. The dosage is as per the dry basis of the emulsion.
6. Place the flask in a shaker water bath running at 200 rpm and 70° C.
7. Collect 10 g of sample into a tube after 1, 2, 4, 23 and 24 hours.
8. Keep the tube in a water bath at 80° C. for at least 15 minutes to deactivate the enzyme.
9. Centrifuge the tube at 3500 rpm for 15 minutes to separate the oil from the water.
10. Collect the Oil for free fatty acid (FFA) analysis.
B. Separation of FFA from the Oil (Lab Neutralization Process)
1. Weigh out about 50 g of hydrolyzed fat.
2. Heat up to 70° C. with continuous stirring.
3. Add a small excess of 4N NaOH to titrate the measured content of FFA in the oil:
The dosage of base that is needed to titrate the measured FFA content was calculated using a MW of 40 for NaOH and MW of 256 for FFA (as if all was palmitic acid). For instance, a 10% FFA content is calculated to require 9.8% w/w dosage of 4N NaOH.
An excess of 1.25% w/w dosage of 4N NaOH was added on top of the amount calculated from the FFA content.
4. Keep stirring for 15 minutes.
5. Centrifuge for 15 minutes at 3500 rpm to separate the fatty acid-soaps from the oil.
6. Collect the oil as the upper layer and add 10% of sodium sulphate before filtering with a membrane filter. This will help to remove water and residual soap from the oil.

### 7. Analyze the FFA and Fatty Acid Composition of the oil

8. Collect the soap and the bottom layer for to do the acidification of soap stock.

### C. Acidification of Soap Stock

1. A strong acid (HCl) was added into the soap stock until there is separation of water. The pH at this moment should be around 2.
2. Heat up the material, and stir it at 80° C. to 90° C. until a clear separation is seen.
3. Collect the FFA of the acid oil on the top layer.

### D. Determination of Fatty Acid Profile

The preparation of Methyl Esters of Fatty Acid is performed according to the method of AOCS Ce2-66. Determination of Fatty Acids in Edible Oils and Fats by Capillary GC as per AOCS Ce1e-91, using an Agilent 6820 Gas Chromatograph with Supelco SP 2340 Fused Silica Capillary Column.

### E. Fatty Acid Content:

FFA is determined for the three starting materials, for each reaction product and for each FFA-free reaction product (after saponification of the sample treated 24 h) using standard autotitrafor methods. As the FFA content is determined on a molar basis (by titration), a representative Mw of the fatty acids is needed to convert to mass basis. To determine FFA in the starting material, the most abundant fatty acid species in the starting material is used: for conversion (C16:0 for Palm stearine; C18:1 for high oleic oil, and C18:2 for soybean oil. To determine FFA in the treated material, Mw for palmitic acid is used as this is the most representative fatty acid of the cleaved fatty acids.

### Results:

TABLE 1
% FFA of High Oleic Oil after Fat Hydrolysis Process

| Time | % FFA |
|---|---|
| 0 hr | 1.90 |
| 1 hr | 5.5 |
| 2 hr | 7.5 |
| 4 hr | 9.8 |
| 23 hr | 14.6 |
| 24 hr | 14.8 |

TABLE 2
Fatty acid composition of Oleic Oil

| | Feed | FFA-free oil |
|---|---|---|
| C12 | — | — |
| C14 | 3.2 | 1.4 |
| C16 | 11.4 | 9.3 |
| C18 | 2.0 | 1.4 |
| C18:1 | 68.0 | 71.6 |
| C18:2 | 8.6 | 8.8 |
| C18:3 | 3.3 | 4.5 |
| C20 | 1.9 | 0.1 |
| Unknown | 1.7 | 2.9 |
| Total Saturation | 18.5 | 12.2 |
| Total Unsaturation | 78.5 | 84.9 |
| Total Monounsaturated | 68.0 | 71.6 |

It is the content of the saturated fatty acids which has been reduced. The unreacted glyceride fraction is enriched in monounsaturates from 68.0% to 71.6%.

TABLE 3
% FFA of Soybean Oil after Fat Hydrolysis Process

| Time | % FFA (as C16) |
|---|---|
| 0 hr | 0.062 |
| 1 hr | 5.4 |
| 2 hr | 7.1 |
| 4 hr | 9.5 |
| 23 hr | 15.3 |
| 24 hr | 15.4 |

TABLE 4
Fatty acid composition of Soybean Oil

| Feed | FFA-free oil | |
|---|---|---|
| C12 | — | — |
| C14 | 0.1 | 0 |
| C16 | 13.5 | 6.9 |
| C18 | 3.6 | 2.4 |
| C18:1 | 24.5 | 26.8 |
| C18:2 | 51.7 | 56.3 |
| C18:3 | 6.3 | 7.2 |
| C20 | 0.3 | 0.4 |
| Unknown | — | — |
| Total Saturation | 17.2 | 9.3 |
| Total Unsaturation | 82.5 | 90.3 |
| Total Monounsaturated | 24.5 | 26.8 |

It is the content of the saturated fatty acids which has been reduced (e.g. C16, C18). The unreacted glyceride fraction is enriched in monounsaturates from 24.5% to 26.8%.

TABLE 5
% FFA of Palm Stearine after Fat Hydrolysis Process

| Time | % FFA (as C16) |
|---|---|
| 0 hr | 0.15 |
| 1 hr | 6.8 |
| 2 hr | 9.5 |
| 4 hr | 14.8 |
| 23 hr | 33.4 |
| 24 hr | 33.0 |

TABLE 6
Fatty acid composition of Palm Stearine

| | Feed | FFA-free oil |
|---|---|---|
| C12 | 0.1 | 0.21 |
| C14 | 1.2 | 1.3 |
| C16 | 62.1 | 44.5 |
| C18 | 5.0 | 4.3 |
| C18:1 | 25.9 | 41.1 |
| C18:2 | 5.4 | 8.2 |
| C18:3 | 0 | — |
| C20 | 0.3 | 0.4 |
| Unknown | — | — |
| Total Saturation | 68.4 | 50.3 |
| Total Unsaturation | 31.3 | 49.3 |
| Total Monounsaturated | 25.9 | 41.1 |

It is the content of the saturated fatty acids which has been reduced (e.g. C16). The unreacted glyceride fraction is enriched in monounsaturates from 25.9% to 41.1%.

### Example 2

### Process Employing 1,3-Specific Lipolytic Enzymes

In a process which combine enzyme reactions with separation a glyceride product is produced with an enriched content of monounsaturated fatty acids.

100 kg palm olein and 7.2 kg ethanol is used as raw material for an enzyme reaction using a 1,3-specific lipolytic enzyme such as e.g. Lipozyme TL IM to produce a mixture of monoglycerides, diglycerides, glycerol, and ethyl esters of fatty acids. The reaction is carried out at a temperature of 40° C. The molar ratio of palm olein to ethanol is 2:1 meaning that for every mole of free fatty acids in the palm olein half a mole ethanol is used. The reaction proceeds until almost all the ethanol has reacted. The enzyme prefers reaction with the 1- and 3-position. The reaction product comprises ethyl esters mainly from saturated fatty acids.

The mixture is separated by distillation or evaporation into two fractions: (1) an ethyl ester fraction mainly comprising saturated fatty acid ethyl esters, and (2) a glyceride fraction mainly comprising glycerides with mainly unsaturated fatty acids. The ethyl ester fraction (1) is further separated by molecular distillation to obtain: (3) almost pure esters of unsaturated fatty acids and (4) almost pure esters of saturated fatty acids.

The glyceride fraction (2) is mixed with the ethyl ester fraction (3) and a lipolytic enzyme: Lipozyme RM IM or Novozym 435. The reaction is carried out at temperature and pressure conditions (e.g. 40° C. and vacuum) allowing the ethanol to be eliminated during the reaction in which glycerides are formed.

The reaction mixture can be added extra ethyl esters of monounsaturated fatty acid obtained by another process to obtain a total of 3 moles of fatty acids in the form of glycerides and as ethylesters to one mole of glycerol. This process can:

a) Use the glyceride fraction (2) in a reaction catalysed by Novozym 435 with ethanol that converts the glyceride to ethylesters of unsaturated fatty acids and glycerol, followed by separation of the glycerol. The glycerol can be eliminated by centrifugation after excessive amounts of ethanol have been evaporated.

b) Obtain the unsaturated fatty acids from ethanolysis of sunflower oil until almost full conversion of fatty acids and glycerol followed by elimination of the glycerol as mentioned above in (a).

c) Use palm oil (or palm olein or sunflower oil) as raw material and hydrolysing with a lipase specific for un-saturated fatty acids in the glycerides. The enzyme can be Geotrichum candidum B lipase. A highly enriched unsaturated free fatty acid fraction is obtained by evaporation of the reaction mixture after this reaction.

The final product obtain can be purified by deodorization.

### Example 3

### Process Employing Lipolytic Enzymes Selective for Saturated Fatty Acids

In a process which combine enzyme reactions with separation a glyceride product is produced with an enriched content of monounsaturated fatty acids.

100 kg palm olein and 7.2 kg ethanol is used as raw material for an enzyme reaction using a lipase reacting preferably with the saturated fatty acids such as e.g. Candida antarctica lipase A to produce a mixture of monoglycerides, diglycerides, glycerol, and ethyl esters of fatty acids. The reaction is carried out at a temperature of 40° C. The molar ratio of palm olein to ethanol is 2:1 meaning that for every mole of free fatty acids in the palm olein half a mole ethanol is used. The reaction proceeds until almost all the ethanol has reacted. The reaction product comprises ethyl esters mainly from saturated fatty acids.

The mixture is separated by distillation or evaporation into two fractions: (1) an ethyl ester fraction mainly comprising saturated fatty acid ethyl esters, and (2) a glyceride fraction mainly comprising glycerides with mainly unsaturated fatty acids. The ethyl ester fraction (1) is further separated by molecular distillation to obtain: (3) almost pure esters of unsaturated fatty acids and (4) almost pure esters of saturated fatty acids.

The glyceride fraction (2) is mixed with the ethyl ester fraction (3) and a lipolytic enzyme: Lipozyme RM IM or Novozym 435. The reaction is carried out at temperature and pressure conditions (e.g. 40° C. and vacuum) allowing the ethanol to be eliminated during the reaction in which glycerides are formed.

The reaction mixture can be added extra ethyl esters of monounsaturated fatty acid obtained by another process to obtain a total of 3 moles of fatty acids in the form of glycerides and as ethylesters to one mole of glycerol. This process can:

a) Use the glyceride fraction (2) in a reaction catalysed by Novozym 435 with ethanol that converts the glyceride to ethylesters of unsaturated fatty acids and glycerol, followed by separation of the glycerol. The glycerol can be eliminated by centrifugation after excessive amounts of ethanol have been evaporated.

b) Obtain the unsaturated fatty acids from ethanolysis of sunflower oil until almost full conversion of fatty acids and glycerol followed by elimination of the glycerol as mentioned above in (a).

c) Use palm oil (or palm olein or sunflower oil) as raw material and hydrolysing with a lipase specific for un-saturated fatty acids in the glycerides. The enzyme can be Geotrichum candidum B lipase. A highly enriched unsaturated free fatty acid fraction is obtained by evaporation of the reaction mixture after this reaction.

The final product obtain can be purified by deodorization.

### Example 4

For separation of esters of palm olein, a centrifugal molecular distillator from Myers Vacuum with rotor diameter of 3 inches is used. At a pressure of 0.001 mmHg, temperatures ranging from 140° C. to 220° C., and a feed flow rate between 0.25 and 0.9 kg/h. The feed is palm olein treated with specific enzymes as demonstrated in examples 1-3. As the temperature is increased the different fractions are isolated: First Ethyl-palmitate is separated at temperatures from 140° C. to 175° C., after which ethyl-oleate, ethyl-stearate and ethyl-linoleate are separated as a single fraction at temperatures from 180° C. to 200° C. Optionally, the separation is terminated once ethyl-palmitate has been separated. Operations can be scaled up using models provided by Batistella et al. (Chem. Eng. Transactions, vol. 3, pp 569-574, 2003).

1-30. (canceled)

31. A process for producing a glyceride product which is enriched in monounsaturated fatty acids relative to the starting glyceride comprising the steps:
a) reacting a triglyceride and an alcohol in the presence of lipolytic enzyme selective for saturated fatty acids and/or selective for the 1-position, the 3-position or both positions in a glyceride, to provide a fraction A, which is enriched in saturated fatty acid esters, and a fraction B, which is enriched in monounsaturated glycerides; and
b) separating fraction A from fraction B.

32. The process of claim 31, further comprising a step c) reacting fraction B or a sub-fraction thereof with (i) a lipolytic enzyme selective for saturated fatty acids and/or a lipolytic enzyme selective for the 1-position, the 3-position or both positions in a glyceride, or (ii) a lipolytic enzyme which is selective for monoglycerides.

33. The process of claim 31, further comprising removing glycerol.

34. The process of claim 31, wherein the triglyceride comprises at least 30% monounsaturated fatty acids.

35. The process of claim 31, wherein the triglyceride comprises at least 50%, at least 55% monounsaturated fatty acid residues in the 2-position.

36. The process of claim 31, wherein the source of triglyceride is palm oil; peanut oil; soybean oil; rapeseed oil; sunflower oil; olive oil; beef tallow; butter fat; cocoa butter; pork lard; poultry fat or their corresponding olein.

37. The process of claim 31, wherein the alcohol is a C1-C3 alcohol.

38. The process of claim 31, wherein the alcohol is ethanol.

39. The process of claim 31, wherein the lipolytic enzyme selective for saturated fatty acids is selected from Candida antarctica lipase A, Fusarium oxysporum lipase, and variants thereof.

40. The process of claim 31, wherein the lipolytic enzyme selective for the 1-position, the 3-position or both positions is selected from Candida antarctica B lipase, Chromobacterium viscosum, dog gastric lipase, dog pancreatic lipase, Fusarium solani cutinase lipase, guinea pig pancreatic lipase, Human gastric lipase, Humicola lanuginosus lipase, human pancreatic lipase, lipoprotein lipase, Mucor miehei lipase, Pseudomonas aeruginosa lipase, Penicillium camembert lipase, Pseudomonas fluorescens lipase, Pseudomonas glumae lipase, porcine pancreatic lipase, Penicillium simplicissimum lipase, Rhizopus arrhizus lipase, rabbit gastric lipase, Fusarium heterosporum lipase, Candida rugosa lipase, and variants thereof.

41. The process of claim 31, wherein the fraction A enriched in saturated fatty acid esters or a sub-fraction thereof is further purified to obtain a sub-fraction A1 which relative to fraction A is enriched in saturated fatty acid esters, a sub-fraction A2 which relative to fraction A is enriched in monounsaturated fatty acid esters, and optionally a sub-fraction A3 which relative to fraction A is enriched in saturated fatty acid esters and which is different from sub-fraction A1.

42. The process of claim 41, wherein sub fraction A1 essentially being ethylpalmitate; sub fraction A2 essentially being ethyloleate and sub fraction A3 essentially being ethylstearate.

43. The process of claim 41, wherein the sub fraction A1 is at least 80% ethylpalmitate.

44. The process of claim 31, wherein the fraction B enriched in monounsaturated glycerides and/or a sub-fraction thereof is re-esterified with a composition rich in monounsaturated fatty acid present as esters or free fatty acids, to produce a glyceride product having at least 70% monounsaturated fatty acids.

45. The process of claim 44, wherein the re-esterification is enzymatic.

46. The process of claim 44, wherein the monounsaturated fatty acid esters for re-esterification are obtained from the sub fraction A2, the sub fraction A2*, or a distillate, hydrolysate or alcoholysate of a vegetable oil.

47. The process of claim 46, wherein the vegetable oil is selected from sunflower oil, peanut oil rapeseed oil, soybean oil, olive oil or alternatively from modified varieties thereof enriched in monounsaturates and/or reduced in polyunsaturates.

48. The process of claim 44, wherein the content of triglycerides in the glyceride product is at least 70%.

49. The process of claim 44, wherein unreacted esters or fatty acids are reused for re-esterification.

50. A glyceride product obtainable by the process of claim 31, comprising at least 70 mole % monounsaturated fatty acids of the total fatty acids in the glycerides.
